(19) United States
(12) Patent Application Publication
(10) Pub. No.: US 2025/0375584 A1
(43) Pub. Date: Dec. 11, 2025

(54) ENDOTRACHEAL TUBE TAPE AND METHOD OF USE THEREOF

(71) Applicant: Jana Petelle, Jamestown, RI (US)

(72) Inventor: Jana Petelle, Jamestown, RI (US)

(21) Appl. No.: 19/235,390

(22) Filed: Jun. 11, 2025

Related U.S. Application Data
(60) Provisional application No. 63/658,585, filed on Jun. 11, 2024.

(51) Int. Cl. A61M 16/04

(52) U.S. Cl. CPC A61M 16/0497

(57) ABSTRACT

A support for an ETT includes a strip of non-rigid material having a first end portion and a second end portion. A main body is disposed between the first end section and second end portion. A first adhesive is disposed on the first end portion. A second adhesive is disposed on the second end portion. A tear line is formed between at least one of the first end portion and the main body and the second end portion and the main body.

### CROSS-REFERENCE TO RELATED APPLICATION

This application claims the benefit of provisional patent application No. 63/658,585 filed Jun. 11 2024, the entire disclosure of which is hereby incorporated in its entirety.

### BACKGROUND OF THE INVENTION

The present invention relates to the structure and use of an endotracheal tube (“ETT”) or laryngeal mask (“LMA”) and more particularly a strap for securing the ETT or LMA in place during surgery.

ETT and LMA are breathing devices that are necessary for airway protection and control of the patient's breathing. These devices are usually placed into the patient at the beginning of the anesthesia process. Once an ETT or LMA is placed, it needs to be secured. It is extremely important that these breathing devices are safely secured in place in a timely and effective manner.

As known in the art, ETTs and LMAs (collectively ETT going forward) are simply secured via standard, generic surgical tape. Securing something so important with simple tape, suffers from several disadvantages. Standard, off the shelf generic surgical tape, does not always stick to the patient's skin, especially when the skin is moist due to sweat, blood, exhalation and/or other bodily fluids allowing the ETT to move. Additionally, skin is not always intact, especially with trauma patients. In older adults, skin can often be too fragile to support tape adhesion without creating skin tears. Lastly, the presence of facial hair, more common now that beards are in fashion for men, makes the use of tape to secure the ETT extremely challenging.

At the end of surgery, it is very important that the ETT is removed in a timely manner when the patient is ready to be extubated. Patients can suffer post-traumatic stress disorder (PTSD) or other complications, from gagging on an ETT while being awakened. Generic surgical tape, suffers from the further disadvantage that it can be very difficult to tear and remove after surgery has been completed causing patient discomfort.

Cross contamination is a serious problem for patients staying in hospitals. The current surgical tape method of ETT positioning suffers from the disadvantage that a roll of tape may be used on one or more patients between operating rooms. This commonly used practice amongst anesthesia providers is not sanitary and increases the risk of cross contamination in already compromised patients. Conversely, the use of one single roll of surgical tape per patient is wasteful, both financially as well as harmful to the environment by increased waste.

Accordingly, there is a need for a structure, methodology, and standardization for securing an endotracheal tube to a patient which overcomes the shortcomings of the prior art.

### SUMMARY OF THE INVENTION

A support for an ETT includes a strip of non-rigid material having a first end portion and a second end portion. A main body is disposed between the first end portion and second end portion. A first adhesive is disposed on the first end portion. A second adhesive is disposed on the second end portion. A tear line is formed between at least one of the first end portion and the main body and the second end portion and the main body.

In one embodiment, a first removeable cover is disposed over the first adhesive. A second removeable cover is disposed over the second adhesive.

In a second embodiment of the invention, the non-rigid material may be formed of a weave, cloth or plastic.

In another embodiment of the invention the tear line is formed as a series of perforations along a height of the main body or as a portion of the strip having a thickness less than the thickness of the end portions sufficient to enable tearing of the end portion from the main body.

In yet another embodiment of the invention, a pad is disposed on the main body and oriented to come in contact with a neck of the user.

In use, the main body of the support for an ETT is positioned against a back of a neck of the patient. The first adhesive portion is secured about the ETT. The second adhesive portion is secured about the ETT. Upon the completion of a procedure, the ETT is removed by first separating one of the first end portion and second end portion from the main body at the tear line.

### BRIEF DESCRIPTION OF THE DRAWINGS

The features and advantages of the present invention will become more readily apparent from the following detailed description of the invention in which like elements are labeled similarly and in which:

FIG. 1A is a top plan view of a structure for securing an ETT to a patient constructed in accordance with the invention; and

FIG. 1B is a side elevation view of a structure for securing an ETT to a patient constructed in accordance with the invention; and

FIG. 1C is a top perspective view of a structure for securing an ETT to a patient constructed in accordance with the invention; and

FIG. 2A is a top plan view of a structure for securing an ETT to a patient, having adhesive covers, constructed in accordance with the invention; and

FIG. 2B is an end view of a structure for securing an ETT to a patient, having adhesive covers, constructed in accordance with the invention; and

FIG. 3 is a top plan view of a structure for securing an ETT to a patient constructed in accordance with a second embodiment of the invention; and

FIG. 4 is another top plan view of the structure for securing an ETT to a patient constructed in accordance with a second embodiment of the invention; and

FIG. 5A is a perspective view of a first step in the use of the structure for securing an ETT to a patient in accordance with the invention; and

FIG. 5B is a perspective view of an alternative first step in the use of the structure for securing an ETT to a patient in accordance with the invention; and

FIG. 6A is a perspective view of an embodiment of an ETT insertion step in the use of the structure for securing an ETT to a patient in accordance with the invention; and

FIG. 6B is a perspective view of an embodiment of an ETT insertion step in the use of the structure for securing an ETT to a patient in accordance with the invention; and

FIG. 7 is a perspective view of a step for exposing the adhesive at a first end of the strip for the use of the structure for securing an ETT to a patient in accordance with the invention; and

FIG. 8 is a perspective view of a step for exposing the adhesive at a second end of the strip for the use of the structure for securing an ETT to a patient in accordance with the invention; and

FIG. 9 is a perspective view of an ETT securing step in the use of the structure for securing an ETT to a patient in accordance with the invention; and

FIG. 10 is a perspective view of an ETT securing step in the use of the structure for securing an ETT to a patient in accordance with the invention; and

FIG. 11 is a perspective view of an ETT securing step in the use of the structure for securing an ETT to a patient in accordance with the invention; and

FIG. 12 is a perspective view of an ETT securing step in the use of the structure for securing an ETT to a patient in accordance with the invention; and

FIG. 13 is a perspective view of an ETT securing step in the use of the structure for removing the ETT from a patient in accordance with the invention; and

FIG. 14 is a perspective view of an ETT securing step in the use of the structure for removing the ETT from a patient in accordance with the invention; and

FIG. 15 is a perspective view of an ETT securing step in the use of the structure for removing the ETT from a patient in accordance with the invention; and

FIG. 16 is a perspective view of an ETT securing step in the use of the structure for removing the ETT from a patient in accordance with the invention; and

FIG. 17 is a perspective view of an ETT securing step in the use of the structure for removing the ETT from a patient in accordance with the invention.

FIG. 18 is a perspective view of an ETT securing step in the use of the structure for removing the ETT from a patient in accordance with the invention.

While the invention is subject to various modifications and alternative forms, specific embodiments thereof are shown by way of example in the specification and claims including the drawings and the accompanying detailed description. It should be understood, however, that the drawings and detailed description are not intended to limit the invention to the particular embodiment which is described. This disclosure is instead intended to cover all modifications, equivalents and alternatives falling within the scope of the present invention as defined by the appended claims. Further, the drawings may not be to scale, and may exaggerate one or more components in order to facilitate an understanding of the various features described herein.

### DETAILED DESCRIPTION OF THE PREFERRED EMBODIMENTS

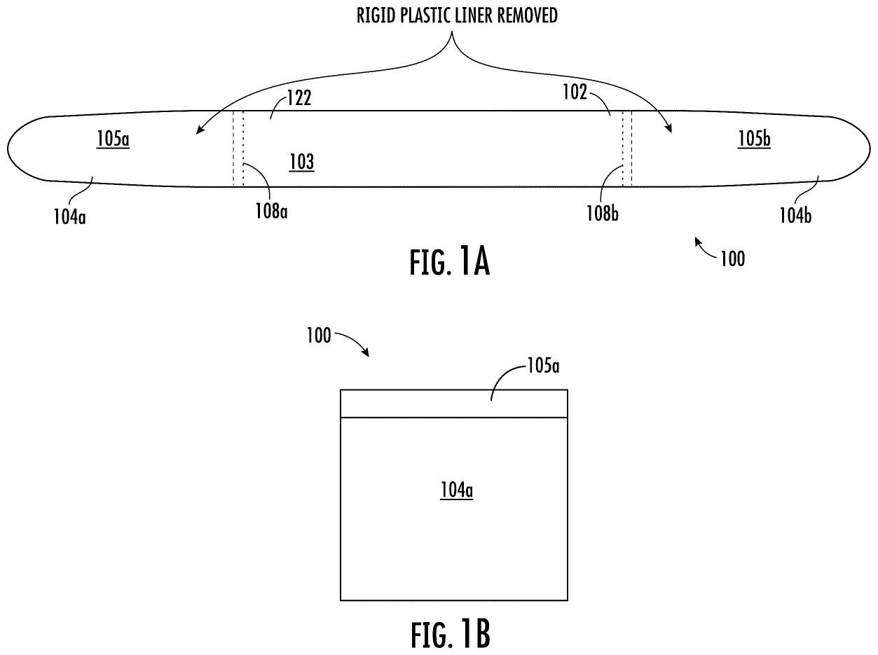

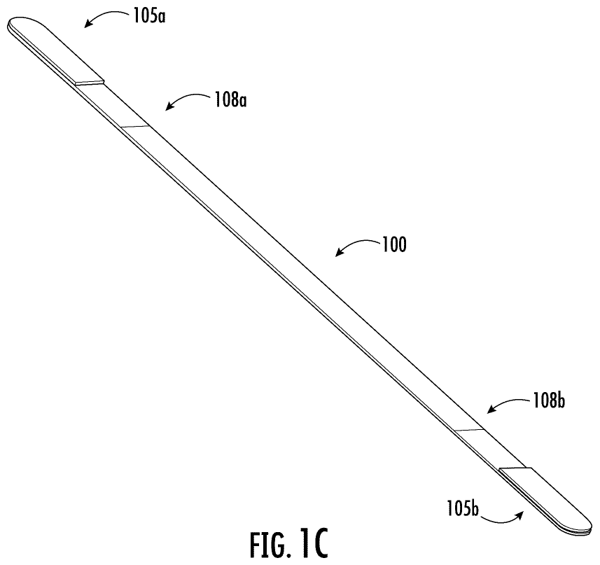

Reference is initially made to FIG. 1 in which a structure, generally indicated as 100, for securing an ETT to a patient, constructed in accordance with the invention is provided. A support for an ETT 100 having a main body 102 which includes a strip 122 of non-rigid material having a first end portion 104a and a second end portion 104b. The main body 102 is disposed between the first end portion 104a and second end portion 104b. A first adhesive 105a is disposed on the first end portion 104a. A second adhesive 105b is disposed on the second end portion 104b. The first end portion 104a and the second end portion 104b may each include rigid or semi-rigid guides (not shown). The guides may be made of HDPE plastic or the equivalent. The adhesive used is a pressure sensitive adhesive known in the art. In a preferred non limiting embodiment, strip 122 is formed from a flexible plastic or cloth tape, preferably silk cloth tape or similar material. Main body 102 is free of adhesive and preferably has a smooth nonstick surface 103. The surface 103 may be an intrinsic part of the main body 102 or may be applied to the main body 102.

A first separation structure such as a tear line 108a is formed between at least one of the first end portion 104a and the main body 102. A second tear line 108b is formed between at least one of the second end portion 104b and the main body 102. Tear lines 108a, 108b may be formed as a line of whole or partial perforations, or the tear lines may be a portion of the support 100 having a narrower thickness than main body 102 and the corresponding adjacent end portion 104a, 104b. In a non-limiting alternative embodiment, only one of the two tear lines 108a or 108b is provided. It may be preferred to have the separation structure to be about six inches from the outer end of the corresponding adjacent end portion.

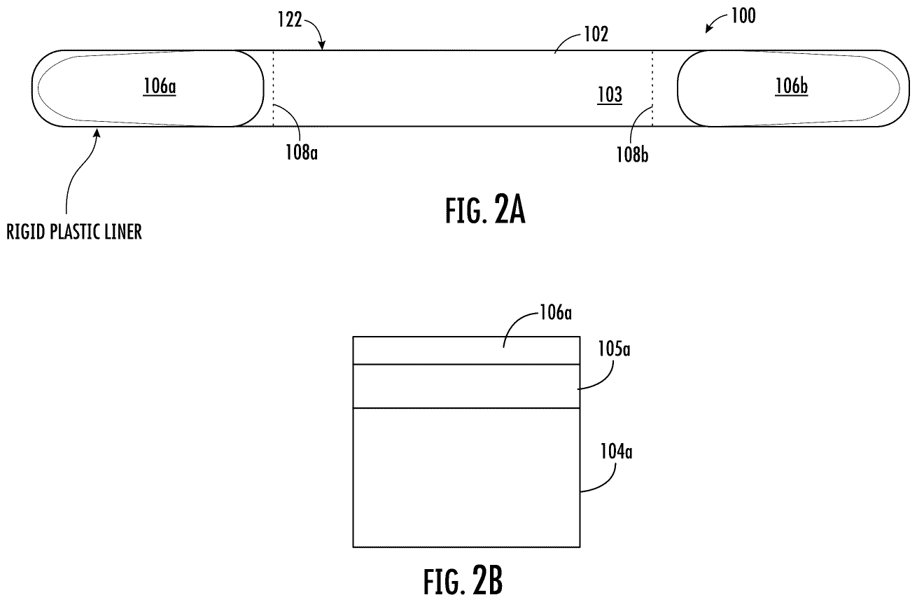

As more particularly seen in FIG. 2, a first removeable cover 106a is disposed on first end portion 104a to cover adhesive 105a. Similarly, a second removeable cover 106b is disposed on second end portion 104b to cover adhesive 105b. As a result of this structure, during transport the adhesive portion is covered to prevent inadvertent sticking to packaging or other nearby structures that may be used for securing an ETT or other purposes in a surgical theater. In a preferred non limiting embodiment, removeable covers 106a, 106b are rigid relative to strip 122. The removable covers 106a, 106b may be made of plastic or a similar material.

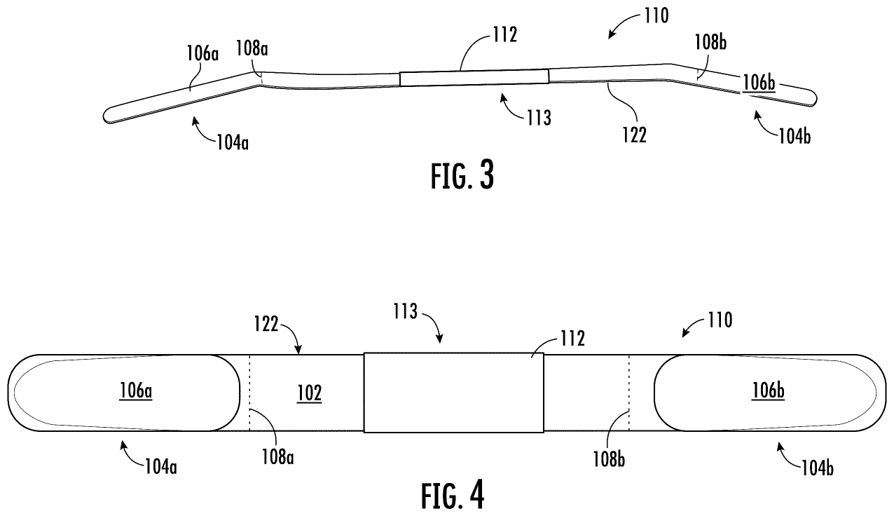

Reference is now made to FIGS. 3 and 4 in which a second embodiment of the invention, generally indicated as 110, is provided, in which like numerals are used to indicate like structure, the primary difference being the inclusion of a cushion 112 for engaging the neck of the patient. A support for an ETT 110 includes a strip 122 of non-rigid material having a first end portion 104a and a second end portion 104b. A main body 102 is disposed between the first end portion 104a and second end portion 104b. A first adhesive 105a is disposed on the first end portion 104a. A second adhesive 105b is disposed on the second end 104b. A first removeable cover 106a is disposed on first end portion 104a to cover adhesive 105a (not shown). Similarly, a second removeable cover 106b is disposed on second end portion 104b to cover adhesive 105b (not shown).

A support pad 112 is disposed on a central portion 113 of the main body 102 and is oriented to come in contact with and cushion the neck and/or head of a patient. Preferably the support pad 112 faces in the same direction as adhesives 105a, 105b. The support pad 112 may be made of cotton batting or an equivalent material.

In a preferred non limiting embodiment support pad 112 for an ETT 100 is sized to fit about a variety of heads, including both adults and children. Support pad 112 for an ETT 100 has a length of approximately thirty to thirty-three inches in a preferred non limiting embodiment. In a preferred non limiting embodiment end portions are sized to secure themselves sufficiently and securely to the ETT as will be shown below. End portions 104a, 104b extend distally from the support pad 112 a length sufficient to engage ETT during use; up to approximately six inches in a preferred nonlimiting embodiment. In a preferred embodiment adhesive 105a, 105b may be coextensive with, or less than the length of end portions 104a, 104b respectively. Removable covers 106a, 106b are at least coextensive with adhesive 105a, 105b respectively. Support pad 112 is disposed to come in contact with the neck of the patient during use and has a length of about six inches in a preferred non limiting embodiment of the invention.

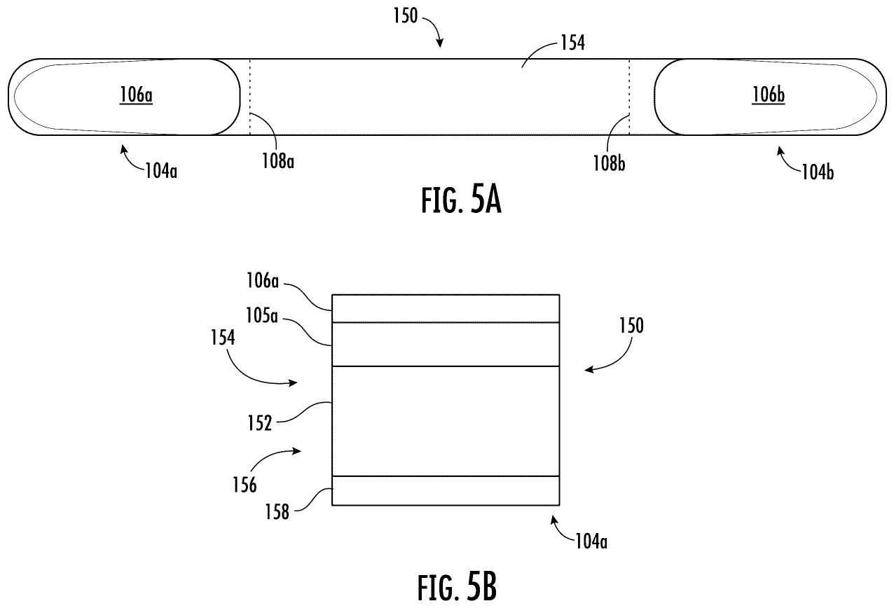

Reference is now made to FIGS. 5a and 5b in which another embodiment of the invention which may be preferred, generally indicated as 150, is provided, in which like numerals are used to indicate like structure, the primary difference being the body of the support 150 is made from a foam or equivalent material and a backing element for rigidity.

For this embodiment, the main body 152 is an elongated member comprised of a flexible material such as closed cell foam such as Roger's foam or its equivalent. The main body 152 has a top portion 154 and a bottom portion 156. Attached to the bottom portion 156 of the main body is a rigid structure 158. The rigid structure 158 may be made from a material such as Mylar PET. The rigid structure may be attached to the bottom portion 156 of the main body 152 by an adhesive or may be formed with the main body 152. The main body 152 includes a first end 104a and a second end 104b. At the first end 104a and the second end 104b of the main body 152, on the top portion 154 of the main body 152, a first adhesive 105a and a second adhesive 105b, respectively, are applied. In addition, respective removable covers 106a, 106b are applied on the adhesive so that the adhesive is controllably exposed. The removable covers 106a, 106b may be made from HDPE or equivalent.

Furthermore, between the first end 104a of the main body 152 and the main body 152 is a separation structure 108a, 108b. The separation structure may alternatively be located between the second end 104b of the main body 152 and the main body 152. Alternatively, separation structures 108a, 108b may be located in both areas. The separation structure may be a series of perforations or a slit tear through either the main body, the rigid structure, or both, or other means known in the art.

The support 100 may be 24 inches to 40 inches in length for an adult. However, the length may be greater or less if the patient may be an infant, a small or large person, or a small or large animal. The location of the one or more separation structures may be about 6 inches from the outer end of the adjacent corresponding end.

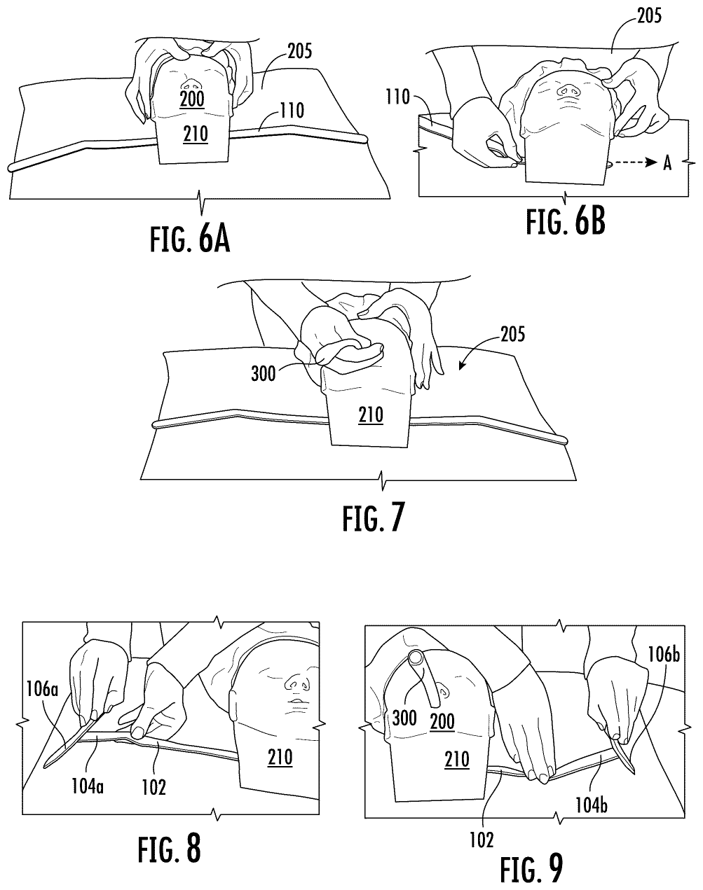

Reference is now made to FIGS. 6a-12 in which a method for using the support for an ETT 100 in accordance with the invention is provided. As seen in FIG. 6a, a head 200 of a patient 205 is placed upon support for an ETT 100; preferably adjacent to the neck 210 of the patient. Adhesives 105a, 105b are oriented to be facing in the same direction as the mouth face of the patient 205. In FIGS. 6a through 18; the direction of the face is up. Alternatively, as shown in FIG. 6b, the support for an ETT 100 may be slid in the direction of Arrow A beneath head 200 of the user adjacent to the neck 210. Alternatively, the support for an ETT 100 may be on the opposite side of the patient 205 as shown in FIG. 6b and slid in the opposite direction of Arrow A beneath head 200 of the user adjacent to the neck 210.

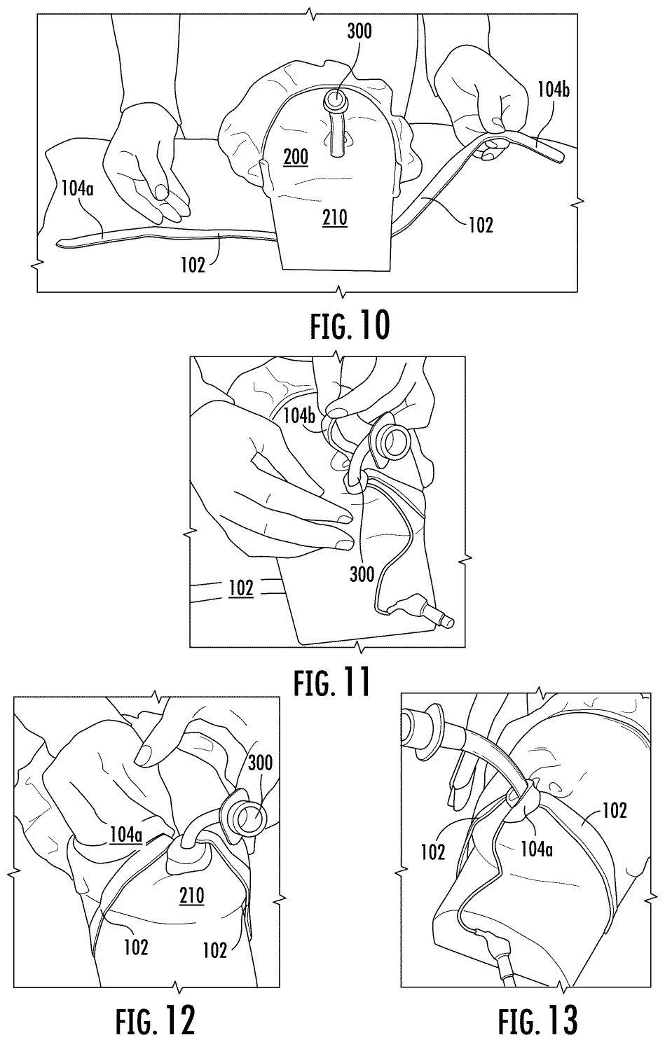

As seen in FIG. 7 an endotracheal tube 300 is placed in the throat of the patient. A first cover 106a is removed from first end portion 104a of strip 122 as shown in FIG. 8. Next, as shown in FIG. 9, a second cover 106b is removed from second end portion 104b of strip 122. As seen in FIG. 10, a first adhesive portion is gripped; the gripped portion is adhesive portion 104b in this non limiting example. As seen in FIG. 11, adhesive portion 104b is secured to endotracheal tube 300 by affixing adhesive portion 104b about the endotracheal tube 300. Then, as seen in FIG. 12, the other adhesive portion (104a in this non-limiting case) is also secured to endotracheal tube 300 by affixing adhesive portion 104a about endotracheal tube 300. As a result, endotracheal tube 300 is anchored in place by strip 122 about the head and/or neck of the patient and affixed in two directions by strip 122.

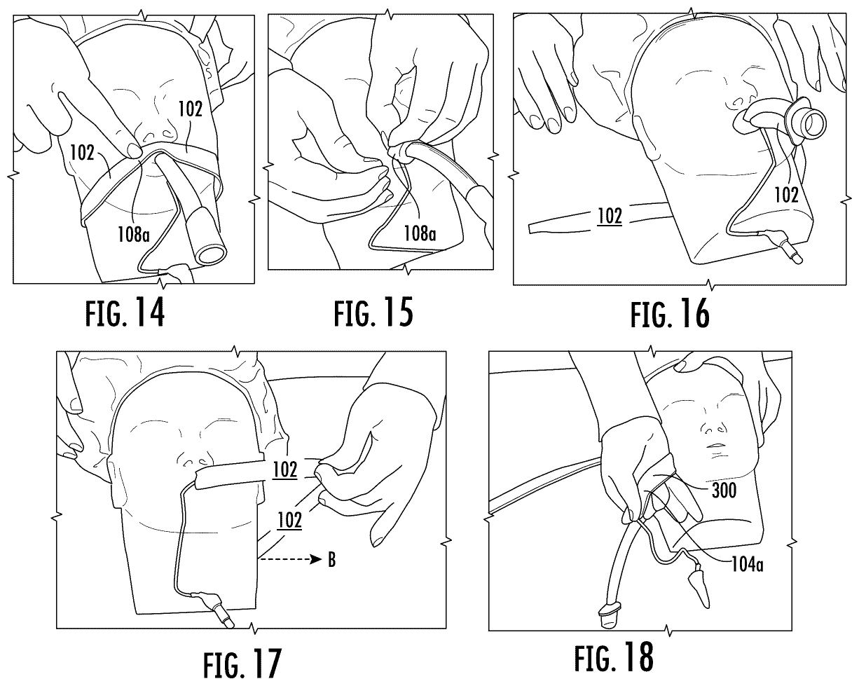

As seen in FIG. 14 and FIG. 15, by applying pressure to strip 122 at a tear line (tear line 108a in this example, strip 122 separates from the end portion 104a between end portion 104a and main body 102. Main body 102 is then slid in the direction of arrow B as seen in FIG. 17, thereby clearing the support for an ETT 100 from the head 200 of the patient. The endotracheal tube 300 is then removed, taking the remnants of the support for an ETT 100 secured thereto at end portion 104b with it; facilitating disposal of both items together as shown on FIG. 18.

As can be seen from the above, once this device has been placed behind the patient's head, the two covers are removed exposing the adhesive. This adhesive can now be used to quickly and securely attach the ETT into place. Again, it is important to note that this new ETT securing device secures an ETT by attaching itself directly to the ETT device without adhering to the patient's skin or hair. Once surgery is finished and the patient needs to be extubated, this device can be removed quickly and safely by easily tearing the perforated section which allows for the fast and safe release of the ETT.

The present ETT securing device, constructed in accordance with the invention, provides a new level of excellence, not only in the practice of anesthesia but also in emergency, trauma, and field medicine. There are many advantages to be considered when using this device over generic, surgical tape. First and foremost, patient safety.

It is incredibly important that the ETT be secured in a timely manner, otherwise it can be dislodged or displaced. As described above, an ETT can be secured quickly and safely due to the device's firm, hard backing material. Its placement and securing to an ETT can be accomplished within seconds. This provides an incredible advantage, when an ETT has to be secured immediately in urgent situations. Traditional devices require full skin prep and presence of a respiratory therapist for its placement. The above-described device allows for a fast placement, which is important when it comes to the safety of the patient.

One of the most challenging issues for quickly and safely securing an ETT, is the condition of the patient's skin. This is another advantage in which the above-described device will allow for an ETT to be securely attached in seconds. The inventive device will secure an ETT safely, regardless of bodily fluids, facial hair, and quality of the skin which makes it ideal for burn victims, trauma patients, patients with facial fractures, and other facial malformations as well as older adults with fragile skin. In addition to skin condition, some patients will have allergic reactions to tape adhesive when placed directly onto their skin. This device simply eliminates that issue.

Yet another advantage to this device, is its size and weight. There are other devices currently being used in the medical field, mostly in intensive care unit (ICU) patients, which are going to be intubated for longer periods of time. Those devices tend to be bigger and more expensive. The described structure lends itself to being lighter and significantly less expensive than those traditional devices, while being even more effective. Additionally, its smaller size makes it ideal to be stored and made accessible directly on a glide scope or other equipment that is being used to place an ETT.

Another significant advantage to our device being smaller and lighter than rolls of tape or traditional devices, is that it makes it an ideal candidate for use in military, field, trauma, and emergency medicine situations. These can often be high-risk, emergency situations where every second counts. Additionally, it will make carrying in the field, where every ounce counts, much easier.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims appended hereto.
should be further recognized that the invention is not limited to the particular embodiments described above. Accordingly, numerous modifications can be made without departing from the spirit of the invention and scope of the claims appended hereto.

What is claimed is:

1. A support for an ETT, comprising:
a strip of non-rigid material having a first end portion and a second end portion; a main body is disposed between the first end section and second end portion;
a first adhesive is disposed on the first end portion;
a second adhesive is disposed on the second end portion; and
a first tear line is formed between at least one of the first end portion and the main body and the second end portion and the main body.

2. The support of claim 1, wherein the first tear line is formed between the first end portion and the main body, and further comprising a second tear line formed between the second end portion and the main body.

3. The support of claim 1, further comprising a support pad disposed upon the main body.

4. The support of claim 1, further comprising first removable cover disposed upon the first adhesive; and a second removable cover disposed upon the second adhesive.

5. The support of claim 4, wherein the first tear line is located between the first removable cover and the main body, and the second tear line is located between the second removable cover and the main body.

6. The support of claim 4, wherein the main body comprises silk cloth.

7. A support for an ETT, comprising:
a strip of flexible material having a first end portion and a second end portion and a top portion and a bottom portion;
a rigid support attached along a length of the strip at the bottom portion of the strip;
a first adhesive disposed on a top portion of the first end portion;
a second adhesive disposed on the second end portion; and
a first separation structure formed between at least one of the first end portion and the main body and the second end portion and the main body.

8. The support for an ETT of claim 7, further comprising:
a second separation structure formed between at least one of the first end portion and the main body and the second end portion and the main body.

9. The support for an ETT of claim 7, wherein the first separation structure comprises one of:
a tear line, a slit tear and perforations.

10. The support for an ETT of claim 7, wherein the second separation structure comprises one of: a tear line, a slit tear and perforations.

11. A method for supporting an ETT in a patient using a support having a strip of flexible material having a first end portion and a second end portion and a top portion and a bottom portion; a rigid support attached along a length of the strip at the bottom portion of the strip; a first adhesive disposed on a top portion of the first end portion; a second adhesive disposed on the second end portion; and an activatable first separation structure formed between at least one of the first end portion and the main body and the second end portion and the main body.
comprising the steps of:
positioning the main body against a back of a neck of the patient;
positioning the first adhesive on the first end portion and the second adhesive on the second end portion in the same direction as a mouth of a patient;
securing the first adhesive portion about the ETT; and
securing the second adhesive portion about the ETT.

12. The method of claim 11, further comprising the steps of:
activating the separation structure.

13. The method of claim 12, wherein the separation structure comprises one of: a tear line, a slit tear and perforations.
